(19) United States
(12) Patent Application Publication
(10) Pub. No.: US 2010/0136083 A1
(43) Pub. Date: Jun. 3, 2010

(54) Metal Implants

(75) Inventors: Thomas Campbell Prentice, Hampshire (GB); Martin Edward Lee Pickford, Hants (GB); David Richard Lewis, Oxfordshire (GB); Andrew Derek Turner, Oxon (GB)

Correspondence Address: Tod T. Tumey, P.O. BOX 22188, HOUSTON, TX, 77227-2188, US

(73) Assignee: ACCENTUS PLC, London (GB)

(21) Appl. No.: 12/521,564

(22) PCT Filed: Jan. 15, 2008
(86) PCT No.: PCT/GB08/50028

§ 371 Date: Jun. 26, 2009

(30) Foreign Application Priority Data
Jan. 15, 2007 (GB) 0700713.1
Feb. 5, 2007 (GB) 0702040.7

(51) Int. Cl. A61K 9/00; B05D 3/14; A61K 33/38; A61P 43/00

(52) U.S. Cl. 424/423; 427/2.26; 424/618

(57) ABSTRACT

An implant with a metal structure for use in a surgical procedure, in which at least part of the metal structure is coated with a biocompatible metal such as titanium by plasma spraying of the metal powder. Biocidal metal cations are then absorbed by ion exchange into the coating, so that after being implanted the biocidal ions gradually leach out into the surrounding body fluids and suppress infection. The ion exchange properties of the coating may be modified by pretreatment with dilute phosphoric acid.

This invention relates to metal implants for use in surgical procedures where the implant is to be at least in partly in contact with bone, and in particular to the introduction of a biocidal material into such implants to suppress or control infection, and to a method of making such implants.

Various surgical procedures require the use of implants. A relatively common surgical procedure of this type is hip replacement wherein the head of the femur is partially or fully replaced to remedy-imperfections due to wear or disease. In another procedure cancerous bone may be removed, in prosthetic surgery, to be replaced by a metal implant. Such implants may for example be of titanium alloy, which is very strong and relatively light. If part of the implant is to be movable relative to adjacent parts of the body then it is known to provide a smooth and polished surface on that part; and where part of the implant is to be embedded in bone it is known to provide a roughened surface to enhance bone growth onto the implant. A suitably roughened surface may be attained by providing a thermally sprayed coating containing hydroxyapatite to enhance growth of bone on to the implant, and/or by plasma-spraying powdered metal onto the surface.

A potential problem with any such implant is the risk of infection. As described in WO 2005/087982 a titanium metal implant can be treated to form a surface layer that is integral with the metal substrate and which incorporates a biocidal material. The method comprises anodising the implant in phosphoric acid at a voltage above 50 V for a period of at least 30 minutes, so as to generate a surface layer, and then performing ion exchange so as to incorporate ions of a biocidal metal into the surface layer. In instances where an implant is to be located in a region where bone growth is not required (e.g. in contact with or in the vicinity of moving muscles), the surface is preferably polished prior to the anodising treatment. Anodising with the specified electrolyte and specified current density generates a hard surface coating of titania typically of thickness about 0.14 μm, but in which there are pits of diameter up to about 5 μm and depths from about 0.4-3 μm which are filled with titanium oxide (or titanium phosphate). Silver ions can then be incorporated, primarily in the material in these pits, to provide the required biocidal effect. A method of introducing such silver ions while avoiding the need for an anodising step would be advantageous.

According to the present invention there is provided an implant, the implant comprising a metal structure, wherein at least part of the surface of the implant incorporates a coating of a biocompatible metal deposited by plasma-spraying a powder of the biocompatible metal, and the coating incorporates biocidal metal cations in the coating.

The present invention also provides a method of making an implant, the implant comprising a metal structure, the method comprising the steps of plasma spraying a powder, of a biocompatible metal on to at least part of the surface of the metal structure so as to form a coating of the biocompatible metal, and then contacting the coating with a solution containing a biocidal metal, such that cations of the biocidal metal are incorporated into the coating.

It is believed that the biocidal cations are incorporated by an ion-exchange process.

That a metal surface deposited by plasma spraying should have ion exchange properties is surprising. It is hypothesised that the ion exchange properties are due to oxide originally on the surface of the individual particles of the powder prior to its deposition. Sufficient biocidal ions can be adsorbed to provide the requisite biocidal effect for a prolonged period, for example for at least six weeks and more preferably for at least six months after implantation, with a low release rate to avoid toxic effects on body cells.

Depending on the material of which the metal structure is made, the plasma-coated structure may then be anodised prior to contact with the solution containing the biocidal metal. It has been found that the anodised coated structure generally incorporates decreased amounts of biocidal metal.

In a modification of the process the deposited coating is first contacted with phosphoric acid (which may convert at least some surface oxide to phosphate); the surface is then rinsed before being contacted with the solution containing the biocidal metal. The rinsing step removes displaceable phosphate ions. The phosphate acid-treated surface generally incorporates decreased amounts of biocidal metal.

In principle, a range of different materials may be used for the biocidal material. Gold, platinum and palladium would be potentially suitable, although expensive; silver is preferable as it is not particularly soluble in body fluids owing to the presence of chloride ions and the low solubility of silver chloride.

The loading of silver ions in the coating of the finished implant may be in the range of from 1 to 100 μg/cm², e.g., from 2 to 20 μg/cm².

Other elements such as copper, tin, antimony, lead, bismuth and zinc might be used as ions combined into the surface layer. The rate of release would be controlled, in this case, primarily by the strength of the bonding forces of the metal ions in the layer.

The metal structures of such prosthetic implants are typically of a form of stainless steel, a titanium alloy, a cobalt/chromium alloy, or alloys or metals based on niobium, tantalum or zirconium. Suitable standard alloys for prosthetic implants are titanium 90% with 6% aluminium and 4% vanadium (British standard 7252), or chromium 26.5-30%, molybdenum 4.5-7%, and the remainder cobalt (British standard 7252 part 4). The coating may be of a titanium alloy, or it may be a pure metal such as titanium, niobium or tantalum, or alloys of these metals.

The biocompatible metal powder may be oxidatively pre-treated prior to the plasma spraying step in order to increase the oxide content of the plasma-sprayed coating. An increase in the oxide content of the plasma-sprayed coating has been found to increase the amount of biocidal metal which can be incorporated into the surface of the coating. The oxidative pre-treatment may be thermal (e.g. heating in air or low O₂/N₂ mixtures under controlled conditions) or by chemical methods such as exposure to an oxidant in solution.

The invention will now be further and more particularly described, by way of example only.

An implant for use as a tibia prosthesis comprises a structure made of cobalt/chromium alloy. The implant structure is of dimensions that are specific for use with a particular patient. At least part of the implant will, when implanted, be in contact with bone, and it is therefore desirable that bone should bond to the surface of that part. This bone bonding process may be helped by providing a rough surface, for example by plasma spraying of titanium powder.

This plasma spraying is a conventional process. It would typically use titanium powder of particle size in the range 30 to 200 μm. To prevent significant oxidation of the powder (which is highly reactive when hot) the plasma spraying would typically use an argon/2% hydrogen plasma, and the powder is sprayed at high velocity through this plasma to impact with the surface. The spraying process may take place in an evacuated chamber, but would more typically be carried out in a chamber containing air. The plasma gases prevent significant contact between the air in the chamber and the hot metal powder, although there may be small amounts of adventitious oxygen which would react with the deposited metal.

If the metal structure of the implant is titanium, niobium, tantalum, zirconium or a suitable alloy of these metals, the plasma-sprayed implant may be anodised in the known manner after the plasma spraying step. It has been found that anodised plasma-sprayed implants incorporate increased amounts of biocidal metal.

Alternatively, the plasma-sprayed implant may be contacted with dilute phosphoric acid to convert at least some of the surface titanium oxide to phosphate, and then preferably rinsed to remove excess phosphate ions.

The coated implant is then contacted with a solution containing silver cations so that these are adsorbed into the oxide/phosphate surface. Preferably, suitable steps may be taken to promote wetting of the coated implant by the silver salt solution. For example, a vacuum may be applied to the solution during contact with the coated implant to displace air bubbles trapped in surface asperities, and/or a non-ionic surfactant may be added to or included in the silver salt solution. The silver salt solution may be stirred to promote uniform incorporation of silver cations in the surface of the plasma-coated implant. In one example, after soaking in 20 wt % phosphoric acid for 2 hours, and then immersion in 0.01 M silver nitrate for 2 hours, the silver loading was 2.4 μg/cm².

In a slightly simpler process the pretreatment with phosphoric acid is not performed, the as-deposited coating being contacted with the solution of the silver salt. This simpler process can also enable sufficient silver to be incorporated. Immersion in 0.01M silver nitrate for 2 hours gave a subsequent silver loading of 3.7 μg/cm², while a similar immersion in 0.05M silver nitrate gave a loading of 13.3 μg/cm².

After the implant is implanted into a patient, silver ions are gradually leached out into the surrounding body fluids, so that any bacteria in the immediate vicinity of the implant are killed. Infection arising from the implant is therefore suppressed.

It will be appreciated that the surface that is coated by plasma spraying with titanium may be pretreated in various ways, for example it would typically be thoroughly cleaned, and may also be shot blasted to provide a rough surface to provide a strong bond.

In a further modification the treated titanium coating (containing biocidal metal, e.g. silver ions) might then be coated with hydroxyapatite, which may be further treated with a solution containing biocidal metal (e.g. treated with dilute silver salt solution) to load or incorporate additional biocidal metal ions, such as silver ions, into the hydroxyapatite.

As described above the titanium coating may be deposited on to a structure of cobalt/chromium alloy, but the titanium coating may equally be deposited on to structures of other metals including titanium alloy. And the coating itself may also be of other metals, for example of cobalt/chromium alloy.

What is claimed:

1. An implant, the implant comprising a metal structure, wherein at least part of the surface of the implant incorporates a coating of a biocompatible metal deposited by plasma-spraying a powder of the biocompatible metal, and the coating incorporates biocidal metal cations in the coating.

2. The implant of claim 1 wherein the biocidal metal cations comprise silver ions.

3. An implant as claimed in claim 2 wherein the loading of silver ions in the coating is from 1 to 100 μg/cm².

4. An implant as claimed in claim 1 wherein the plasma coated structure is either anodized or treated with phosphoric acid.

5. The implant of claim 1 wherein the plasma-coated structure containing biocidal metal is coated with hydroxyapatite.

6. A method of making an implant, the implant comprising a metal structure, the method comprising the steps of plasma spraying a powder of a biocompatible metal on to at least part of the surface of the metal structure so as to form a coating of the biocompatible metal, and then contacting the coating with a solution containing a biocidal metal, such that cations of the biocidal metal are incorporated into the coating.

7. A method as claimed in claim 6 wherein the coating is first contacted with phosphoric acid before being contacted with the solution containing the biocidal metal.

8. A method as claimed in claim 6 wherein the coated structure is anodized prior to contacting with the solution of biocidal metal.

9. A method as claimed in claim 6 wherein the plasma-coated structure containing biocidal metal is coated with hydroxyapatite.

10. A method as claimed in claim 6 wherein the biocidal metal is silver.

11. A method as claimed in claim 5 wherein additional biocidal metal cations are loaded into the hydroxyapatite.

12. A method as claimed in claim 9 wherein the hydroxyapatite-coated structure is treated with a solution containing biocidal metal cations to incorporate at least some of the latter biocidal metal cations into the hydroxyapatite coating.
